(19) United States
(12) Patent Application Publication
(10) Pub. No.: US 2019/0100547 A1
(43) Pub. Date: Apr. 4, 2019

(54) METHOD FOR PREPARING 2'-O-FUCOSYLLACTOSE

(71) Applicant: BASF SE, Ludwigshafen am Rhein (DE)

(72) Inventors: MICHAEL PUHL, Ludwigshafen am Rhein (DE); Stephanie RENZ, Ludwigshafen am Rhein (DE); Andreas WOELFERT, Ludwigshafen am Rhein (DE)

(21) Appl. No.: 16/082,581

(22) PCT Filed: Mar. 8, 2017
(86) PCT No.: PCT/EP2017/055403

§ 371 Date: Sep. 6, 2018

(30) Foreign Application Priority Data
Mar. 9, 2016 (EP) 16159434.6

(51) Int. Cl. C07H 3/08; C07H 1/00

(52) U.S. Cl. CPC C07H 3/08; C07B 51/00; C07H 1/00

(57) ABSTRACT

The present invention relates to a method for preparing 2′-O-fucosyllactose and to the protected fucosyl donor of the formula (I) used in this method. The method comprises reacting the fucose derivative of the formula (I) below with the compound of the general formula (II), in the presence of an activating reagent. In the formulae (I) and (II), the variables are each defined as follows: X is Br or a S-bound radical, namely —SCN, —S(O)ₙ—R^{X1}or —S—R^{X2}, wherein R^{X1} preferably is an optionally substituted phenyl, and R^{X2} preferably is C₁-C₄-alkyl, 2-oxazolin-2-yl, 2-thiazolin-2-yl, benzoxazol-2-yl, benzothiazol-2-yl or pyridin-2-yl; R^{Si} are the same or different and are radicals of the formula SiR^{a}R^{b}R^{c}, wherein R^{a}, R^{b} and R^{c} preferably are each methyl; R¹ is a C(=O)—R¹¹ radical or an SiR¹²R¹³R¹⁴ radical, wherein R¹¹ is preferably methyl, phenyl or tert-butyl, and R¹², R¹³ and R14 preferably are each methyl; R² are the same or different and are C₁-C₈-alkyl or together form a linear C₃-C₆-alkanediyl, which is unsubstituted or has 1 to 6 methyl groups as substituents; R³ are the same or different and are C₁-C₈-alkyl or together form a linear C₁-C₄-alkanediyl, which is unsubstituted or has 1 to 6 methyl groups as substituents.

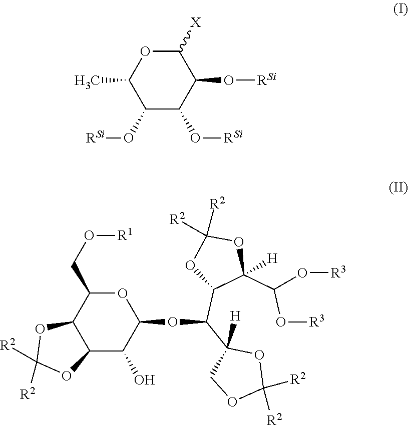

The present invention relates to a new method for preparing 2′-O-fucosyllactose and to the protected fucosyl donor used in this method.

### BACKGROUND OF THE INVENTION

2′-O-fucosyllactose (CAS-No.: 41263-94-9: α-L-fucopyranosyl)-(1→2)-O-β-D-galactopyranosyl-(1→4)-D-glucopyranose) is an oligosaccharide, which is found in relatively large quantities in breast milk. It has been variously reported that the 2′-O-fucosyllactose present in breast milk causally reduces the risk of infection in newborns who are breast fed (see e.g. Weichert et al, Nutrition Research, 33 (2013), Volume 10, 831-838; Jantscher-Krenn et al, Minerva Pediatr. 2012, 64 (1) 83-99; Morrow et al, J. Pediatr. 145 (2004) 297-303). 2′-O-fucosyllactose is therefore of particular interest as a constituent of food supplements, particularly as additive for humanized milk products, particularly for infant nutrition.

The preparation of 2′-O-fucosyllactose by classical chemical or biochemical means has been variously described in the literature (see e.g. Carbohydrate Res. 88(1) (1981) 51, Carbohydrate Res. 154 (1986) 93-101, Carbohydrate Res. 212 (1991) C1-C3, J. Org. Chem. (1997) 62, 992, Heterocycles 84(1) (2012) 637, U.S. Pat. No. 5,438,124, WO 2010/115934, WO 2010/115935, WO 2010/070616, WO 2012/113404 and WO 2013/48294). The chemical preparation is typically based on the fucosylation of suitably protected acceptors, i.e. lactose derivatives that are partially protected, bearing the only unprotected hydroxyl group in the 2-position of the galactosyl moiety, e.g. 4-O-(6-O-acetyl-3,4-isopropylidene-β-D-galactopyranosyl)-2,3;5,6-bis-O-isopropylidene-D-glucose dimethylacetal, with activated fucosyl donors which bear e.g. a thioalkyl group, an alkenyloxy group, a trichloroacetimidate or a bromine atom in place of the anomeric OH group, such as methyl 1-thio-2,3,4-tri-O-benzyl-β-L-fucopyranoside, methyl 3,4-O-isopropylidene-2-O-(4-methoxybenzyl)-1-thio-L-fucopyranoside, pentenyl 3,4-O-isopropylidene-2-O-(4-methoxybenzyl)-β-L-fucopyranoside, phenyl 1-thio-2,3,4-tri-O-benzyl-β-L-fucopyranoside, 2,3,4-tri-O-benzyl-β-L-fucopyranosyl bromide, or 2,3,4-tri-O-benzyl-β-L-fucopyranosyl trichloroacetimidate (with respect to fucose donors see the literature cited above and Tetrahedron Lett. 31 (1990) 4325). A particular disadvantage is that the benzyl protecting groups of the fucosylating reagents must be removed by hydrogenolysis using heavy metal-containing catalysts, which leads to impurities in the product that are difficult to remove and unacceptable for foodstuff.

For instance, R. K. Jain et al., Carbohydrate Research, 212 (1991), pp. C1-C3, describe a route for the preparation of 2′-O-fucosyllactose by fucosylation of 4-O-(6-O-acetyl-3,4-isopropylidene-β-D-galactopyranosyl)-2,3; 5,6-bis-O-isopropylidene-D-glucose dimethylacetal using methyl 3,4-O-isopropylidene-2-O-(4-methoxybenzyl)-1-thio-β-L-fucopyranoside or pentyl 3,4-O-isopropylidene-2-O-(4-methoxybenzyl)-β-L-fucopyranoside as fucosylating reagents. These fucosylating reagents are, however, complex to prepare and require hydrogenolytic debenzylation after the fucosylation step. A similar synthesis is described in J. Org. Chem. 1997, 62, 992.

WO 2010/115934 and WO 2012/113404 describe the preparation of 2-fucosyllactose using 2-O-benzylated fucosyl donors. The preparation of the fucosyl donors is rather complex and, in addition, the protected trisaccharide obtained following the fucosylation step requires hydrogenolytic deprotection. A similar method is known from WO 2010/070616.

Ott et al., J. Carbohydr. Chem. 2001, 20 (7&8), 611-636, describe inter alia the synthesis of 2′-L-fucosyllactose analogues using a trisilylated fucosyl building block which, however, is modified with a spacer-moiety.

Thus, the fucosylation methods known to date typically result in 2′-O-fucosyllactose containing impurities which cannot be removed completely, such as transition material and aromatics from the hydrogenolytic removal of benzyl protecting groups, and also undesirable trisaccharides, such as the β-isomer of 2′-O-fucosyllactose, namely β-L-fucopyranosyl-(1→2)-O-β-D-galactopyranosyl-(1→4)-D-glucopyranose. These impurities are particularly problematic, if 2′-O-fucosyllactose is used in human nutrition, in particular infant nutrition.

### SUMMARY OF THE INVENTION

It is an object of the present invention to provide a method for preparing 2′-O-fucosyl-lactose which does not involve the problems of the prior art. The method should in particular allow the use of starting materials that can be easily prepared, particularly readily available fucosyl donors. The method should further ensure good yields and good stereoselectivity in the fucosylation. In addition, the method should be suitable so as to avoid the removal of any protecting groups by hydrogenolysis over transition metal catalysts.

It has been found that by reacting trisilylated fucose derivatives having an anomeric leaving group, which function as fucosyl donors and are of the formula (I) below,

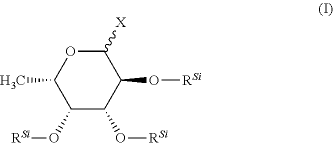

where

R^{Si} are suitable silyl protecting groups, and

X is a suitable leaving group,

with appropriate lactose acceptors, namely the compounds of the general formula (II) defined in more detail below, in the presence of an activator, the corresponding protected 2′-O-fucosyllactose derivatives of the general formula (III) are obtained in good yields and high selectivity, which can then be deprotected in a manner known per se to obtain 2′-O-fucosyllactose, without a hydrogenation step being required.

Accordingly, the invention firstly relates to a method for preparing 2′-O-fucosyllactose, comprising the steps of:

a) reacting the fucose derivative of the general formula (I)

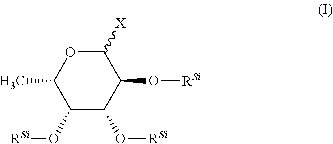

where
R^{Si} are the same or different radicals of the formula SiR^{a}R^{b}R^{c},, in which R^{a}, R^{b} and R^{c} are the same or different and are selected from C₁-C₈-alkyl, C₃-C₈-cycloalkyl, phenyl and C₃-C₈-cycloalkyl-C₁-C₄-alkyl;
X is selected from the group consisting of Br and S-bound radicals, namely —SCN, —S(O)ₙ—R^{X1} or —S—R^{X2}, where
   n is 0, 1 or 2,
   R^{X1} is aryl which is unsubstituted or optionally has 1 to 5 substituents selected from halogen, C₁-C₄-alkyl, C₁-C₄-alkoxy, C₁-C₄-haloalkyl and C₁-C₄-haloalkoxy, and
   R^{X2} is selected from the group consisting of C₁-C₆-alkyl, C₁-C₆-haloalkyl, benzyl, wherein the phenyl moiety of benzyl is unsubstituted or optionally has 1 to 5 substituents selected from halogen, C₁-C₄-alkyl, C₁-C₄-alkoxy, C₁-C₄-haloalkyl and C₁-C₄-haloalkoxy, and 5- or 6-membered heterocyclyl, which bears a nitrogen atom in ortho position relative to the point of attachment and optionally a second heteroatom selected from O and S in the other ortho position, where heterocyclyl may optionally carry a fused benzyl moiety;

with a compound of the general formula (II)

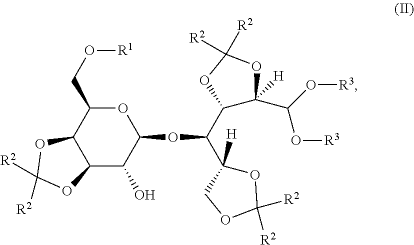

where
R¹ is a radical C(═O)—R¹¹ or an radical SiR¹²R¹³R¹⁴, in which
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-alkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄-alkyl;
R² may be the same or different and are C₁-C₈-alkyl or two radicals R² attached to the same carbon atom together form a linear C₃-C₆-alkanediyl, which is unsubstituted or has 1 to 6 methyl groups as substituents;
R³ may be the same or different and are C₁-C₈-alkyl or together form a linear C₁-C₄-alkanediyl, which is unsubstituted or has 1 to 6 methyl groups as substituents; and

b) deprotecting the coupling product of the general formula (III) obtained in step a),

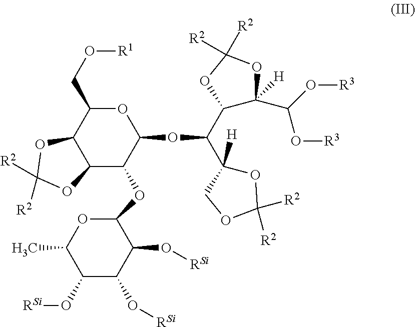

where R^{Si}, R¹, R² and R³ are as defined above;

to obtain 2′-O-fucosyllactose.

The compound of formula (III) may be deprotected in step b) of the inventive method by removing all protecting groups in one step or, alternatively, by successive removal of the protecting groups in two or more steps. In the latter case, the following partially protected 2′-O-fucosyllactose derivatives of the general formulae (IIIa), (IIIb) and (IIIc) may be obtained as intermediates:

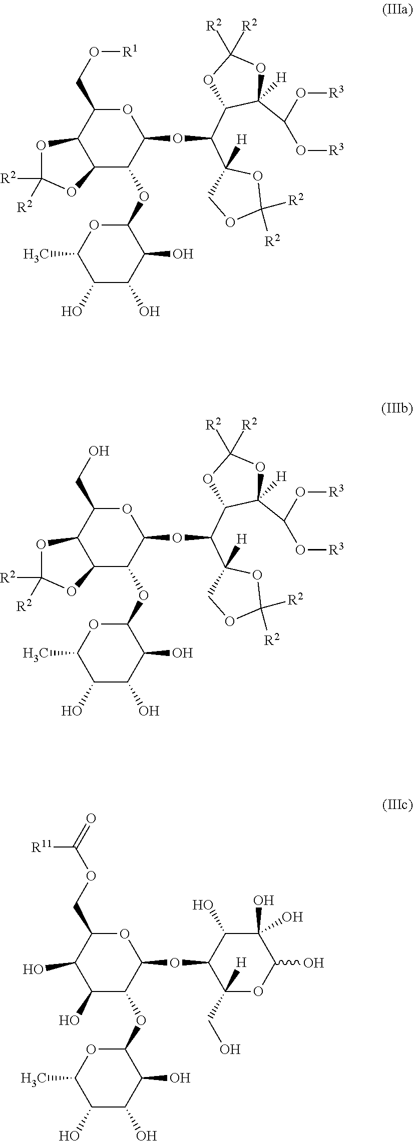

in which R¹, R², R³ and R¹¹ are as defined for formula (II).

The invention further relates to 2,3,4-trisilylated fucosyl donors with a Br radical or an S-bound radical as an anomeric leaving group. In this regard, 1-(4-methyl-thiophenyl)-2,3,4-O-trimethylsilyl-L-fucopyranose is known from Y.-C. Ko et al., J. Am. Chem. Soc., 2014, 136 (41), 14425-31, 1-sulfinylphenyl-2,3,4-O-triethylsilyl-L-fucopyranose is disclosed in U.S. Pat. No. 5,700,916, 1-thioethyl-2,3,4-O-triethylsilyl-L-fucopyranose and 1-thioethyl-2,3,4-O-tri-(tert-butyldimethylsilyl)-L-fucopyranose are known from R. Daly et al., J. Org. Chem. 2013, 78 (3), 1080-90, and 1-bromo-2,3,4-O-tri-(tert-butyldimethylsilyl)-L-fucopyranose is also known from the prior art. Accordingly, the invention particularly relates to fucose derivates of the general formula (I), where
R^{Si} are the same or different radicals of the formula SiR^{a}R^{b}R^{c}, in which
   R^{a}, R^{b} and R^{c} are the same or different and are selected from C₁-C₈-alkyl, C₃-C₈-cycloalkyl, phenyl and C₃-C₈-cycloalkyl-C₁-C₄-alkyl;
X is selected from the group consisting of Br and S-bound radicals, namely —SCN, —S(O)ₙ—R^{X1} or —S—R^{X2}, where
   n is 0, 1 or 2,
   R^{X1} is aryl which is unsubstituted or optionally has 1 to 5 substituents selected from halogen, C₁-C₄-alkyl, C₁-C₄-alkoxy, C₁-C₄-haloalkyl and C₁-C₄-haloalkoxy, and
   R^{X2} is selected from the group consisting of C₁-C₆-alkyl, C₁-C₆-haloalkyl, benzyl, wherein the phenyl moiety of benzyl is unsubstituted or optionally has 1 to 5 substituents selected from halogen, C₁-C₄-alkyl, C₁-C₄-alkoxy, C₁-C₄-haloalkyl and C₁-C₄-haloalkoxy, and 5- or 6-membered heterocyclyl, which bears a nitrogen atom in ortho position relative to the point of attachment and optionally a second heteroatom selected from O and S in the other ortho position, where heterocyclyl may optionally carry a fused benzyl moiety;

with the exception of compounds of the formula (I), wherein
R^{Si} is trimethylsilyl and X is —S-(4-methyl-phenyl),
R^{Si} is triethylsilyl and X is —S-ethyl or —S(O)-phenyl, or
R^{Si} is tert-butyldimethylsilyl and X is Br or —S-ethyl.

The inventive method is linked to a series of advantages. The method affords the primary coupling product of the formula (III) in good yields and good stereoselectivity. The removal of the protecting groups in the compound of the formula (III) is possible under mild hydrolysis conditions, without the need for a hydrogenolysis over transition metal catalysts. The resulting intermediates of the formula (III), particularly of the formulae (IIIa) and (IIIb), are stable, in particular stable during storage, and may be purified. In addition, the method can readily be carried out on a relatively large scale. A further advantage is that the 2′-O-fucosyllactose obtainable by the method according to the invention, in comparison to the known 2′-O-fucosyllactose, does not comprise, or only comprises in much lower fractions, those impurities which cannot be removed, for example the heavy metals and heavy metal compounds resulting from a hydrogenation, and also alkyl aromatic compounds which are formed by hydrogenation of benzyl protecting groups. Furthermore, by the method of the invention, the undesirable β-isomer is not formed or only formed to a very low extent, which is much lower than the amount of β-isomer formed in the methods of the prior art. Indeed, by the reaction of the compound of formula (I) with the compound of formula (II), the undesirable β-isomer of the compound of formula (III) is formed in such a low amount that the molar ratio of β-isomer (III-β) to α-isomer (III-α) does not exceed 1:7, and in particular is about 1:10, i.e. is in the range of approximately 1:8 to 1:15. Thus, the method of the invention allows for producing the desired 2′-O-fucosyllactose which, optionally after further purification, contains less than 1.5% by weight, in particular less than 1.0% by weight, of the undesirable β-isomer.

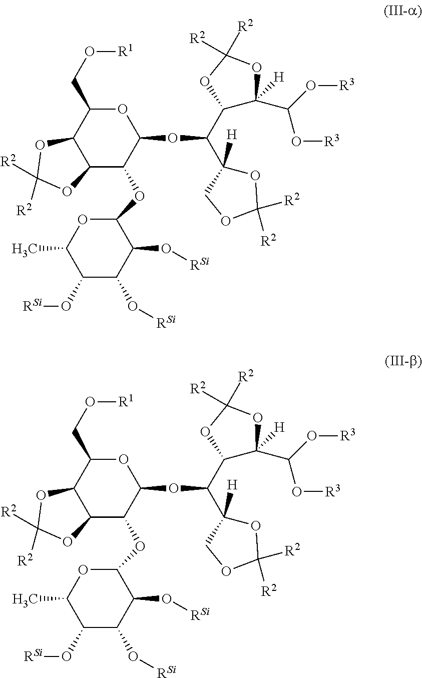

The quality of the 2′-O-fucosyllactose obtained by the method according to the invention renders it particularly suitable for preparing foodstuffs. Accordingly, the present invention also relates to
the 2,3,4-trisilylated fucosyl donors of the formula (I) used in the method described herein;
the 2′-O-fucosyllactose obtainable by the method described here;
the use of the 2′-O-fucosyllactose obtainable by the method described here in foodstuffs or as food additive; and
a foodstuff or food additive, comprising 2′-O-fucosyllactose, obtainable by a method as described herein and at least one carrier suitable for foodstuff.

### DETAILED DESCRIPTION OF THE INVENTION

In the context of the present invention, the terms used generically are defined as follows:

The prefix Cₓ-C_{y} denotes the number of possible carbon atoms in the particular case.

The term “halogen” in each case denotes fluorine, bromine, chlorine or iodine, specifically fluorine, chlorine or bromine.

The term “C₁-C₄-alkyl” denotes a linear or branched alkyl radical comprising 1 to 4 carbon atoms, such as methyl, ethyl, propyl, 1-methylethyl (isopropyl), butyl, 1-methylpropyl (sec-butyl), 2-methylpropyl (isobutyl) or 1,1-dimethylethyl (tert-butyl).

The term “C₁-C₈-alkyl” denotes a linear or branched alkyl radical comprising 1 to 8 carbon atoms. Examples, in addition to the radicals mentioned for C₁-C₄-alkyl, are n-pentyl, n-hexyl, n-heptyl, n-octyl, 2-pentyl, 2-hexyl, 2-heptyl, 2-octyl, 3-pentyl, 3-hexyl, 3-heptyl, 3-octyl, 2,2-dimethylpropyl, 2-methylbutyl, 3-methylbutyl, 2-ethylbutyl, 3-ethylbutyl, 2-methylpentyl, 3-methylpentyl, 4-methylpentyl, 2-ethylpentyl, 3-ethylpentyl, 4-ethylpentyl, 2-ethylhexyl and positional isomers thereof.

The term “C₁-C₈-haloalkyl” denotes a linear or branched alkyl radical comprising 1 to 8 carbon atoms, particularly 1 to 4 carbon atoms (C₁-C₄-haloalkyl), in which one or more or all hydrogen atoms have been replaced by halogen atoms, in particular by fluorine or chlorine atoms. Examples for this purpose are chloromethyl, dichloromethyl, trichloromethyl, fluoromethyl, difluoromethyl, trifluoromethyl, chlorofluoromethyl, dichlorofluoromethyl, chlorodifluoromethyl, 2,2-difluoroethyl, 2,2,2-trifluoroethyl, 1,1,2,2-tetrafluoroethyl, pentafluoroethyl, 2,2-difluoropropyl, 3,3-difluoropropyl, 3,3,3-trifluoropropyl, 2,2,3,3,3-pentafluoropropyl, heptafluoropropyl, and the like.

The term “C₁-C₄-alkoxy” denotes straight-chain or branched saturated alkyl groups comprising 1 to 4 carbon atoms which are bonded via an oxygen atom. Examples of C₁-C₄-alkoxy are methoxy, ethoxy, n-propoxy, 1-methylethoxy (isopropoxy), n-butoxy, 1-methylpropoxy (sec-butoxy), 2-methylpropoxy (isobutoxy) and 1,1-dimethylethoxy (tert-butoxy).

The term “C₁-C₄-haloalkoxy” denotes straight-chain or branched saturated haloalkyl groups comprising 1 to 4 carbon atoms which are bonded via an oxygen atom. Examples in this case are fluoromethoxy, difluoromethoxy, trifluoromethoxy, 1-fluoroethoxy, 2-fluoroethoxy, 2,2-difluoroethoxy, 2,2,2-trifluoroethoxy, 1,1,2,2-tetrafluoroethoxy, pentafluoroethoxy, 3,3,3-trifluoroprop-1-oxy, 1,1,1-trifluoroprop-2-oxy, 1-fluorobutoxy, 2-fluorobutoxy, 3-fluorobutoxy, 4-fluorobutoxy and the like.

The term “C₃-C₈-cycloalkyl” denotes a cyclic, saturated hydrocarbyl radical comprising 3 to 8 carbon atoms. Examples are cyclopropyl, cyclobutyl, cyclopentyl, cyclohexyl, cycloheptyl and cyclooctyl.

The term “C₃-C₈-cycloalkyl-C₁-C₄-alkyl” denotes a linear or branched alkyl radical comprising 1 to 4 carbon atoms, in which one hydrogen atom has been replaced by C₃-C₈-cycloalkyl, as defined above.

The term “linear C₁-C₄-alkanediyl” denotes a linear, divalent hydrocarbyl radical having 1 to 4 carbon atoms, such as methylene, ethane-1,2-diyl, propane-1,3-diyl, and butane-1,4-diyl.

The term “linear C₃-C₆-alkanediyl” denotes a linear, divalent hydrocarbyl diradical having 3 to 6 carbon atoms, such as propane-1,3-diyl, butane-1,4-diyl, pentane-1,5-diyl and hexane-1,6-diyl.

The term “5- or 6-membered heterocyclyl, which bears a nitrogen atom in ortho position relative to the point of attachment and optionally a second heteroatom selected from O and S in the other ortho position, where heterocyclyl may optionally carry a fused benzyl moiety” refers to a 5- or 6-membered saturated, partially unsaturated or aromatic heterocyclic ring bearing in ortho position relative to the attachment point of the heterocyclic ring is to the remainder of the molecule a nitrogen atom, which may or may not substituted with a hydrogen atom. In the other ortho position, the heterocyclic ring optionally bears a second heteroatom selected from O and S. Examples of such heterocyclic rings are 2-pyrrolidinyl, 2-oxazolidinyl, 2-thiazolidinyl, 2-piperidinyl, 1,3-oxazinan-2-yl, 1,3-thiazinan-2-yl, 1-pyrrolin-2-yl, 1-pyrrolin-5-yl, 2-pyrrolin-2-yl, 2-pyrrolin-5-yl, 3-pyrrolin-2-yl, 2-oxazolin-2-yl, 3-oxazolin-2-yl, 4-oxazolin-2-yl, 2-thiazolin-2-yl, 3-thiazolin-2-yl, 4-thiazolin-2-yl, 1,4-dihydropyridin-2-yl, 5,6-dihydro-4H-1,3-oxazin-2-yl, 5,6-dihydro-2H-1,3-oxazin-2-yl, 2,3-dihydro-6H-1,3-oxazin-2-yl, 2,3-dihydro-4H-1,3-oxazin-2-yl, 5,6-dihydro-4H-1,3-thiazin-2-yl, 5,6-dihydro-2H-1,3-thiazin-2-yl, 2,3-dihydro-6H-1,3-thiazin-2-yl, 2,3-dihydro-4H-1,3-thiazin-2-yl, 2H-1,3-oxazin-2-yl, 4H-1,3-oxazin-2-yl, 6H-1,3-oxazin-2-yl, 2H-1,3-thiazin-2-yl, 4H-1,3-thiazin-2-yl, 6H-1,3-thiazin-2-yl, 2-pyrrolyl, 2-oxazolyl, 2-thiazolyl and 2-pyridinyl. These heterocyclic rings may optionally carry a fused benzyl moiety, i.e. the heterocyclic ring and the benzyl moiety share two adjacent carbon atoms. Examples for such heterocyclic rings carrying a fused benzyl moiety are indolin-2-yl, isoindolin-1-yl, benzoxazolin-2-yl, benzthiazolin-2-yl, 1,2,3,4-tretrahydro-chinolin-2-yl, indol-2-yl, isoindol-1-yl, benzoxazol-2-yl, benzthiazol-2-yl, chinolin-2-yl, 2H-1,3-benzoxazin-2-yl and 2H-1,3-benzthiazin-2-yl.

The term “foodstuff” or “food” denotes compositions and formulations which are intended and suitable as nutrition for mammals, particularly human beings. In the context of the present invention, they include both compositions based on naturally-occurring products, e.g. dairy products, and also artificially prepared formulations, for example, for dietary or medicinal nutrition, which can be used directly or optionally have to be converted into a ready-to-use formulation before use by addition of liquid.

The term “food additive” denotes substances which are mixed with the foodstuff to achieve chemical, physical or also physiological effects.

With respect to the method according to the invention and the compounds of the formulae (II), (III), (IIIa) and (IIIb), the variables R² within one formula preferably have the same definition in each case. R² is in particular C₁-C₄-alkyl and especially methyl or two radicals R² attached to the same carbon atom are together 1,5-pentanediyland thus form a cyclohexane-1,1-diyl residue with the carbon atom to which they are attached. All radicals R² are especially methyl.

With respect to the method according to the invention and the compounds of the formulae (II), (III), (IIIa) and (IIIb), the variables R³ within one formula preferably have the same definition in each case. R³ is particularly C₁-C₄-alkyl and especially methyl.

With respect to the method according to the invention and the compounds of the formulae (I) and (III), the variables R^{Si} within one formula preferably have the same definition in each case. R^{Si} is particularly tri(C₁-C₄-alkyl)silyl, especially trimethylsilyl, i.e. in the SiR^{a}R^{b}R^{c} radical, the radicals R^{a}, R^{b} and R^{c} are the same or different and are particularly C₁-C₄-alkyl, especially methyl.

A preferred first embodiment of the present invention relates to a method, where in the compound of the formula (I) the radical X is Br.

A preferred second embodiment relates to a method, where in the compound of the formula (I) the radical X is an S-bound radical different from Br that is preferably —S—R^{X1} or —S—R^{X2}, wherein
R^{X2} is phenyl, which is unsubstituted or optionally has 1, 2 or 3 substituents selected from halogen, C₁-C₄-alkyl and C₁-C₄-alkoxy, and
R^{X2} is selected from the group consisting of C₁-C₄-alkyl, 2-oxazolin-2-yl, 2-thiazolin-2-yl, benzoxazol-2-yl, benzothiazol-2-yl and pyridin-2-yl.

In other words, according to this embodiment the radical X is preferably selected from the group consisting of C₁-C₄-alkylthio, 2-oxazolin-2-ylthio, 2-thiazolin-2-ylthio, benzoxazol-2-ylthio, benzothiazol-2-ylthio, pyridin-2-ylthio and phenylthio, wherein the phenyl moiety is unsubstituted or optionally has 1, 2 or 3 substituents selected from halogen, C₁-C₄-alkyl and C₁-C₄-alkoxy. In addition, according to this embodiment, the radical X is especially selected from C₁-C₄-alkylthio and phenylthio, wherein the phenyl moiety is unsubstituted or optionally has 1, 2 or 3 substituents selected from Br, Cl, C₁-C₄-alkyl and C₁-C₂-alkoxy, and especially is methylthio, ethylthio or phenylthio.

A third embodiment relates to a method, where in the compounds of the formulae (II) and (III) the radical R¹ a siR¹²R¹³R¹⁴ radical, particularly tri(C₁-C₄-alkyl)silyl, especially trimethylsilyl, i.e. in the SiR¹²R¹³R¹⁴ radical, the radicals R¹², R¹³ and R¹⁴ are the same or different and are particularly C₁-C₄-alkyl, especially methyl. In accordance to this embodiment, the radical R¹ in the formula (IIIa) is tri(C₁-C₄-alkyl)silyl, especially trimethylsilyl.

A fourth preferred embodiment relates to a method, where in the compounds of the formulae (II) and (III) the radical R¹ is a —C(═O)—R¹¹ radical, wherein R¹¹ is as defined above and is particularly hydrogen, C₁-C₄-alkyl, C₁-C₄-haloalkyl or phenyl, which may be substituted with one or two substituents selected from halogen, methyl and ethyl, especially is methyl, tert-butyl, phenyl, 4-chlorophenyl or 4-methylphenyl and specifically is methyl, tert-butyl or phenyl. Consequently, in this embodiment, the radical R¹ is especially acetyl, pivaloyl, benzoyl, 4-chlorobenzoyl or 4-methylbenzoyl, and specifically acetyl, pivaloyl or benzoyl. In the context of this embodiment, also the radical R¹¹ in the formula (IIIc) has the same meanings mentioned above as preferred.

In certain embodiments of the invention, R¹¹ differs from methyl. In special groups of embodiments, R¹¹ is methyl. In further special groups of embodiments, R¹¹ is tert-butyl or phenyl.

An example of a particularly preferred compound of the formula (I) is the compound of the formula (I), where all radicals R^{Si} are trimethylsilyl, and the radical X is Br.

Examples of further particularly preferred compounds of the formula (I) are
the compound of the formula (I), where all radicals R^{Si} are trimethylsilyl, and the radical X is methylthio;
the compound of the formula (I), where all radicals R^{Si} are trimethylsilyl, and the radical X is ethylthio; and
the compound of the formula (I), where all radicals R^{Si} are trimethylsilyl, and the radical X is phenylthio.

In the method of the invention, the compound of formula (I) may in principle be employed in the form of its α-anomer (I-α) or of its β-anomer (I-β) or, alternatively, in the form of a mixture of its α-anomer (I-α) and its β-anomer (I-β).

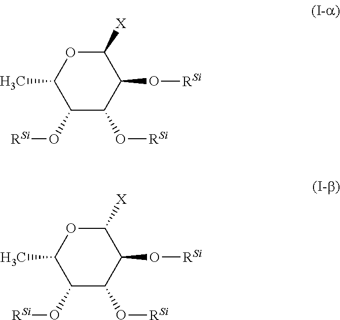

However, the anomericity of the compound I used in the inventive method does usually not noticeably affect the anomericity of the newly formed glycosidic bond, i.e. whether the linkage of the fucosyl moiety in the compound of formula (III) is in the α- or the β-configuration.

An example of a particularly preferred compound of the formula (II) is the compound of the formula (II), where all radicals R² are methyl, all radicals R³ are methyl, and R¹ is trimethylsilyl.

An example of a further particularly preferred compound of the formula (II) is also the compound of the formula (II), where all radicals R² are methyl, all radicals R³ are methyl, and R¹ is acetyl.

Another example of a further particularly preferred compound of the formula (II) is also the compound of the formula (II), where all radicals R² are methyl, all radicals R³ are methyl, and R¹ is benzoyl.

Another example of a further particularly preferred compound of the formula (II) is also the compound of the formula (II), where all radicals R² are methyl, all radicals R³ are methyl, and R¹ is pivaloyl, i.e. —C(═O)—C(CH₃)₃.

Examples of particularly preferred compounds of the formula (III) are
the compound of the formula (III), where all radicals R² are methyl, all radicals R³ are methyl, all radicals R^{Si} are trimethylsilyl, and R¹ is trimethylsilyl;
the compound of the formula (III), where all radicals R² are methyl, all radicals R³ are methyl, all radicals R^{Si} are trimethylsilyl, and R¹ is acetyl;
the compound of the formula (III), where all radicals R² are methyl, all radicals R³ are methyl, all radicals R^{Si} are trimethylsilyl, and R¹ is pivaloyl.

Examples of particularly preferred compounds of the formula (IIIa), with R¹ being a —C(═O)—R¹¹ radical, are
the compound of the formula (IIIa), with R¹=—C(═O)—R¹¹, where all radicals R² are methyl, all radicals R³ are methyl, and R¹¹ is methyl;
the compound of the formula (IIIa), with R¹=—C(═O)—R¹¹, where all radicals R² are methyl, all radicals R³ are methyl, and R¹¹ is phenyl;
the compound of the formula (IIIa), with R¹=—C(═O)—R¹¹, where all radicals R² are methyl, all radicals R³ are methyl, and R¹¹ is tert-butyl.

An example of a particularly preferred compound of the formula (IIIb) is
the compound of the formula (IIIb), where all radicals R² are methyl, and all radicals R³ are methyl.

Examples of particularly preferred compounds of the formula (IIIc) are
the compound of the formula (IIIc), where R¹¹ is methyl;
the compound of the formula (IIIc), where R¹¹ is ethyl;
the compound of the formula (IIIc), where R¹¹ is phenyl;
the compound of the formula (IIIc), where R¹¹ is tert-butyl.

In step a) of the method according to the invention the fucose derivative of the formula (I), which functions as fucosyl donor, is reacted with a compound of the formula (II) in the presence of an activating reagent to give the fucosylated compound of the formula (III). Depending on whether a fucose derivative of formula (I) according to the first or the second embodiment is used, i.e. whether the radical X is Br or an S-bound radical, the fucosylation reaction in step a) is hereinafter also referred to as Process A and Process B, respectively.

For the Processes A and B, in principle, any reagent is suitable as activating reagent that is known in the prior art to promote glycosylations using glycosyl donors which either carry, in the case of Process A, a Br radical or, in the case of Process B, a S-bound radical, as anomeric leaving group. Glycosylations using such glycosyl donors are disclosed for example in the “Handbook of Chemical Glycosylation” edited by Alexei V. Demchenko, 2008, Wiley-VCH Verlag, Weinheim, Germany, and the literature cited therein.

For the fucosylation of Process B, i.e. the reaction of step a) using a compound of formula (I) with X being an S-bound radical, the activating reagent is preferably selected from the following glycosylation promoting reagents:
i) chloramine T, i.e. the sodium salt of N-chloro 4-methylbenzenesulfonamide, as described e.g. in A. K. Misra et al., Carbohydr. Res. 2004, 339, 885-890;
ii) iodonium dicollidine perchlorate, as described e.g. in R. U. Lemieux et al., Canadian Journal of Chemistry 1965, 43, 2205, and G. H. Veeneman et al., Tetrahedron Lett. 1990, 31, 275-278, and Ott et al., J. Carbohydr. Chem. 2001, 20 (7&8), 611-636;
iii) dimethyl(methylthio)sulfonium triflate, as described e.g. in P. Fugedi et al., Carbohydr. Res. 1986, 149, C9-C12, and Ott et al., J. Carbohydr. Chem. 2001, 20 (7&8), 611-636;
iv) N-bromosuccinimide (NBS), as described e.g. in K. C. Nicolaou et al., J. Am. Chem. Soc. 105 (1983), 2430-34;
v) N-iodosuccinimide (NIS);
vi) N-bromosuccinimide plus triflic acid (TfOH), as described e.g. in M. Sasaki et al., Tetrahedron Lett. 1991, 32, 6873-76;
vii) N-bromosuccinimide plus trimethylsilyl triflate (TMSOTf), as described e.g. in Z. H. Qin et al., Carbohydr. Res. 2002, 337, 31-36;
viii) N-iodosuccinimide plus triflic acid, as described e.g. in G. H. Veeneman et al., Tetrahedron Lett. 1990, 31, 1331-34, and P. Konradsson et al., Tetrahedron Lett. 1990, 31, 4313-16;
ix) N-iodosuccinimide plus trimethylsilyl triflate, as described e.g. in G. H. Veeneman et al., Tetrahedron Lett. 1990, 31, 1331-34, and P. Konradsson et al., Tetrahedron Lett. 1990, 31, 4313-16;
x) bromine (Br₂) plus silver(I) triflate (AgTf), as described e.g. in J. O. Kihlberg et al., J. Org. Chem. 1990, 55, 2860-63;
xi) diphenylsulfoxide (Ph₂SO) plus triflic anhydride (TfO₂), as described e.g. in J. D. C. Codee, et al., Tetrahedron 2004, 60, 1057-64, and J. D. C. Codee, et al., Organic Letters 2003, 5, 1519-22;
xii) iodine (I₂) plus hexamethyldisilazane (HMDS), as described e.g. in P. Cura et al., Synlett 2000, 1279-80, and K. P. R. Kartha et al., Tetrahedron: Asymmetry 2000, 11, 581-593; and
xiii) copper(II) bromide (CuBr₂) plus tetra-(C₁-C₆-alkyl) ammonium bromide ((C₁-C₆-alkyl)₄NBr), as described e.g. in J. O. Kihlberg et al., J. Org. Chem. 1990, 55, 2860-63, and S. Sato et al., Carbohydr. Res. 1986, 155, C6-C10.

These reagents or glycosylation promoters are particularly suited for the fucosylations of Process B using a fucose derivative of formula (I) with the radical X being selected from the group consisting of C₁-C₄-alkylthio, 2-oxazolin-2-ylthio, 2-thiazolin-2-ylthio, benzoxazol-2-ylthio, benzothiazol-2-ylthio, pyridin-2-ylthio and phenylthio, and especially from methylthio, ethylthio and phenylthio.

If the activating reagent used in Process B is one of the reagents i) to v), which each consist of a single component, it is usually employed in an amount of 0.05 to 2 molar equivalents, preferably 0.5 to 1.5 molar equivalents and in particular 0.8 to 1.2 molar equivalents per 1 mole of the compound of the formula (I).

If the activating reagent used in Process B is one of the reagents vi) to xiii), which each consist of two different components, it is usually employed in such an amount, so that per 1 mole of the compound of the formula (I) there are 1 to 2 molar equivalents, preferably 1 to 1.5 molar equivalents, of the first mentioned component, i.e. NBS, NIS, Br₂, Ph₂SO, I₂ or CuBr₂, and 0.01 to 2 molar equivalents, preferably 0.05 to 1.5 molar equivalents, of the second mentioned component, i.e. TfOH, TMSOTf, AgTf, TfO₂, HMDS or (C₁-C-alkyl)₄NBr.

In some instances it may be beneficial to use the second mentioned component of the reagents vi) to xiii) in an amount of usually 1 to 2 molar equivalents and in particular 1 to 1.5 molar equivalents per 1 mole of the compound of the formula (I). In other instances, however, it may be beneficial to use the second mentioned component of the reagents vi) to xiii) in an amount of usually 0.01 to 0.25 molar equivalents and in particular 0.05 to 0.1 molar equivalents per 1 mole of the compound of the formula (I).

More preferably, the fucosylation of Process B is carried out in the presence of an activating reagent selected from the promoters iv) to xiii), in particular from the promoters iv) to xi) and specifically from the promoters vi), vii), viii) and ix), i.e. NBS plus triflic acid, NBS plus trimethylsilyl triflate, NIS plus triflic acid and NIS plus trimethylsilyl triflate.

For the fucosylation of Process A, i.e. the reaction of step a) using a compound of formula (I) with X=Br, the activating reagent is a glycosylation promoter that is preferably selected from alkaline earth metal bromides and tetra-(C₁-C₆-alkyl) ammonium bromides, and specifically is tetra-n-butyl ammonium.

In Process A, the activating reagent is typically used in an amount of 0.05 to 2 molar equivalents, preferably 0.5 to 1.5 molar equivalents and in particular 0.8 to 1.2 molar equivalents per mole of the compound of the formula (I).

In step a) of the method of the present invention, i.e. in Process A as well as in Process B, the compounds of the formulae (I) and (II) are reacted with each other in a molar ratio of the compound of the formula (I) to the compound of the formula (II) in the range of generally 1:3 to 3:1, particularly 1:2 to 2:1, particularly preferably 1:1.5 to 1.5:1, and especially 1:1.1 to 1.1:1.

Step a), i.e. the reaction of the fucose derivative of the formula (I) with the compound of the formula (II), is generally carried out in an inert organic solvent or diluent. Preference is given to aprotic solvents, particularly those having a low content of protic impurities, such as water, alcohols or acids. The content of protic impurities in the solvent is preferably less than 1000 ppm. Preferably, before being used in the method according to the invention, the aprotic solvent is treated to reduce the content of protic impurities, particularly water, by treatment with suitable absorbents, for example with molecular sieves of pore size 3 to 4 Angström. Preferred aprotic organic solvents are haloalkanes, such as dichloromethane, trichloromethane, dichloroethane, aromatic hydrocarbons, such as toluene and xylenes, acyclic and cyclic ethers, such as diethyl ether, dimethoxyethane, tetrahydrofurane (THF) and 1,4-dioxane, dimethylamides of aliphatic carboxylic acids, such as dimethylformamide (DMF) and dimethylacetamide, and also alkyl nitriles, such as acetonitrile, and also mixtures of the abovementioned solvents. Particularly preferred aprotic organic solvents are dichloromethane, acetonitrile, DMF, toluene, THF, diethyl ether, dimethoxyethane, 1,4-dioxane and mixtures thereof. In addition, for the reaction in step a) the solvent is preferably selected such that all constituents are present in dissolved form.

The reaction in step a) is preferably carried out at temperatures in the range of −100 to 30° C. and particularly in the range of −20 to 0° C. The reaction may be carried out at ambient pressure, at reduced or elevated pressure. The reaction is typically conducted at a pressure in the range of 900 to 1100 mbar.

The compound of the formula (III) obtained by the reaction in step a) may be isolated by customary work-up methods and optionally be purified by crystallization and/or chromatography. Alternatively, it is possible to directly subject the compound of the formula (III) obtained by the reaction in step a) to at least partial or complete deprotection so as to, thus, obtain one of the compounds of the formulae (IIIa), (IIIb) or (IIIc), or 2′-O-fucosyllactose.

It has surprisingly been found that the fucose derivatives of the formula (I), wherein the variable X is Br, can conveniently be prepared by reacting the corresponding fucose derivatives of the formula (I), wherein X is an S-bound radical and in particular is thiomethyl, thioethyl or thiophenyl, with elemental bromine. For this purpose, bromine is usually used in an amount of 0.8 to 2 moles, preferably 1 to 1.8 moles and in particular 1.1 to 1.5 moles per mol of the fucose derivative of the formula (I) with X being an S-bound radical.

The compound of the formula (I) with X being an S-bound radical is typically reacted with bromine at a temperature in the range of −100 to 40° C., particularly in the range −80 to 10° C. and especially in the range of −20 to 0° C. The reaction may be carried out at ambient pressure or at reduced or elevated pressure. Typically, the reaction is conducted at a pressure in the range of 900 to 1100 mbar.

The reaction of the compound of the formula (I), wherein X is an S-bound radical, with bromine is generally carried out in one of the inert organic solvent or diluents mentioned above. Preference is given here to the aforementioned aprotic solvents, particularly those having a low content, preferably less than 1000 ppm, of protic impurities, such as water alcohols or acids. Particularly preferred solvents in this context are dichloromethane, acetonitrile, DMF, toluene, THF, diethyl ether, dimethoxyethane, 1,4-dioxane and mixtures thereof.

As discussed above, the compounds of formula (I) with X=Br are suitable fucosyl donors to be used in the method of the invention for preparing compounds of the formula (III). Thus, a particular aspect of the invention relates to the inventive method, where step a) comprises reacting the compound of formula (I), wherein the radical X is an S-bound radical different from Br, with bromine to obtain a compound of formula (I), wherein X is Br, followed by reacting the compound of formula (I), wherein X is Br, with the compound of formula (II) in the presence of the activating reagent, to obtain a compound of the formula (III).

The reaction product resulting from the reaction of the compound of the formula (I), wherein X is an S-bound radical, with bromine is preferably not isolated, but is subjected without further isolation or purification to the reaction with a compound of formula (II) in step a) of the inventive method that is discussed in detail herein before. Thus, it is possible to prepare the compound of the formula (I) with X=Br in-situ and then add directly to the reaction mixture obtained this way the compound of formula (II) as well as an activating reagent in order to initiate the fucosylation of step a). Alternatively, the reaction product resulting from the reaction of the compound of formula (I), wherein X is an S-bound radical, with bromine can also be purified or isolated, for example by removing volatile constituents for the reaction mixture, preferably under reduced pressure, and possibly further steps, such as crystallization and/or chromatography.

In step b) of the inventive method, the deprotection of the compound of the formula (III) is achieved in analogy to known deprotecting reactions and is preferably carried out by hydrolysis methods. The conditions for cleavage of these protecting groups are familiar to those skilled in the art, e.g. from P. G. M. Wuts et al., “Greene's Protecting Groups in Organic Synthesis”, 4th Edition, Wiley 2006, and the literature cited therein, or the references cited at the outset for the preparation of 2′-O-fucosyllactose.

According to a first embodiment b.1) of the invention, the compound of the formula (III) is treated with water in the presence of an acid. In this manner, a complete cleavage of all protecting groups from the compound of the formula (III) is generally achieved and the 2′-O-fucosyllactose is obtained.

Suitable acids are mineral acids, such as hydrochloric acid, sulfuric acid, phosphoric acid, acidic salts of mineral acids, such as alkali metal hydrogen phosphates and dihydrogen phosphates or alkali metal hydrogen sulfates, e.g. sodium dihydrogen phosphate or potassium hydrogen phosphate, in addition organic carboxylic acids, such as acetic acid, propionic acid, dichloroacetic acid, trichloroacetic acid or trifluoroacetic acid, and organic sulfonic acids, such as methanesulfonic acid. The acids are typically used as dilute aqueous acids, e.g. as 5 to 70% strength by weight solutions. Frequently, the diluted aqueous acid is used in combination with a suitable organic solvent. Examples thereof are organic solvents miscible with water, such as C₁-C₄-alkanols, e.g. methanol, ethanol, isopropanol, 1-butanol or tert-butanol, cyclic ethers, such as tetrahydrofuran or dioxane, and also organic solvents having only limited miscibility with water, e.g. haloalkanes, such as dichloromethane, trichloromethane, dichloroethane, aromatic hydrocarbons, such as toluene and xylenes, and also dialkyl ethers, such as diethyl ether, diisopropyl ether or methyl tert-butyl ether. The reaction conditions required are known to a person skilled in the art, e.g. from P. G. M. Wuts et al., loc. cit. and the literature cited therein, or the references cited at the outset for the preparation of 2′-O-fucosyllactose. Subsequent to the removal of the protecting groups, the acid is usually neutralized and then the product is isolated by removal of water. Neutralization can be achieved by using a base, which is conventionally used for this purpose, including alkalimetal hydroxides, alkalimetal carbonates and alkalimetal bicarbonates. Neutralization can also be achieved by using a basic or strongly basic ion-exchange resin, because this will allow for neutralization without formation of salts in the solution of the product.

In the embodiment b.1), cleavage of the protecting groups can also be achieved by means of an acidic ion-exchange resin in aqueous media. Thereby, a separate neutralization step can be avoided.

According to a further embodiment b.2) of the invention, the compound of the formula (III), in which R₁ is a —siR¹²R¹³R¹⁴ radical, is firstly treated with a desilylating reagent, wherein a compound of the formula (IIIb) is obtained:

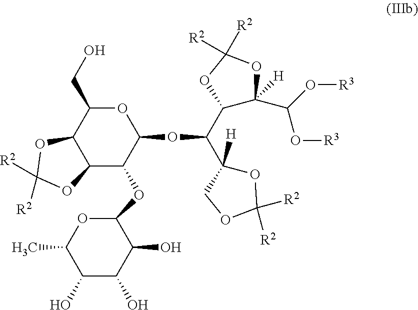

The desilylation may be carried out in one step, such that both the —SiR¹²R¹³R¹⁴ group and the —SiR^{a}R^{b}R^{c} groups are simultaneously cleaved off. It can also be carried out successively if the SiR¹²R¹³R¹⁴ and SiR^{a}R^{b}R^{c} groups have different reactivities.

Suitable reagents for the desilylation are, for example, the abovementioned C₁-C₄ alcohols, particularly methanol, with or without addition of water, and also alkali metal or alkaline earth metal carbonates and hydrogen carbonates, such as lithium carbonate, sodium carbonate, potassium carbonate, sodium hydrogen carbonate and potassium hydrogen carbonate, preferably in solution in one of the abovementioned C₁-C₄ alcohols, particularly methanol, with or without addition of water. Suitable desilylating reagents are also tetraalkylammonium fluorides, which are preferably used in polar, aprotic organic solvents, e.g. cyclic ethers, such as tetrahydrofuran or dioxane, or in di-C₁-C₄-alkylamides of aliphatic carboxylic acids, such as dimethylformamide or dimethylacetamide, or alkyl nitriles, such as acetonitrile or mixtures of the abovementioned polar, aprotic organic solvents. The reaction conditions required are known to a person skilled in the art, e.g. from P. G. M. Wuts et al., loc. cit. and the literature cited therein.

Subsequently, the remaining protecting groups are removed by treating the compound of the formula (IIIb) with water in the presence of an acid. This can be effected in the manner described for embodiment b.1).

According to a further embodiment b.3) of the invention, the compound of the formula (III), in which R¹ is a —C(O)R¹¹ radical, is firstly treated with a desilylating reagent, wherein a compound of the formula (IIIa′) is obtained:

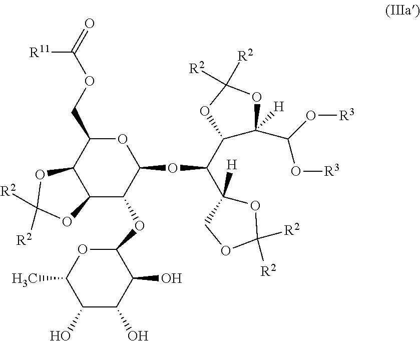

The compound of the formula (IIIa′) corresponds to the compound of the formula (IIIa), where R¹ is a —C(O)R¹¹ radical. Subsequently, the —C(O)—R¹¹ group and the remaining protecting groups are simultaneously or successively removed.

The desilylation of the compound of the formula (III), in which R¹ is a —C(O)R¹¹ radical, is achieved in analogy to embodiment b.2) by treatment of the compound (III) with a desilylating reagent. The reaction conditions required for the desilylation are known to a person skilled in the art, e.g. from P. G. M. Wuts et al., loc. cit. and the literature cited therein.

The subsequent cleavage of the ester group —C(O)—R¹¹ is achieved in a manner known per se by basic saponification or by base-catalyzed or enzyme-catalyzed transesterification. Methods for this purpose are known, e.g. from P. G. M. Wuts et al. loc. cit. or from Kociensky et al., “Protective groups”, 3rd Edition, Chapter 4.6, Thieme 2005. The remaining C(R²)₂ and OR³ protecting groups are then removed in a manner known per se, e.g. by treatment with an aqueous acid, as already described in connection with embodiment b.1).

According to a further embodiment b.4), the procedure can, alternatively, also be such that the C(R²)₂ and OR³ protecting groups are initially removed from the compounds of the formula (IIIa′), e.g. by treatment with an aqueous acid, as already described in connection with embodiment b.1), wherein the compound of the general formula (IIIc) is obtained as previously described. The ester group —C(═O)—R¹¹ can then be cleaved from the compound of the formula (IIIc) in a manner known per se. e.g. by basic saponification or basic transesterification or by enzyme-catalyzed transesterification.

According to a particularly preferred embodiment b.5), the compound of formula (III), in which R¹ is a —C(O)R¹¹ radical, is treated with a C₁-C₄-alkanol and an alkalimetal base first, whereby a compound of formula (IIIb) is obtained, followed by removal of the remaining protective groups under acidic conditions. In this embodiment, R¹¹ is preferably C₁-C₄-alkyl, such as methyl, ethyl or tert-butyl. Thereby, the desilylation and the removal of the ester group —C(O)—R¹¹ can be linked to each other and may be carried in a single step. Suitable reagents here are in turn the above mentioned alkali metal hydroxides and carbonates in C₁-C₄-alkanols, such as methanol, as solvent. For this purpose, the combination of methanol with sodium carbonate or potassium carbonate is particularly useful. The reaction conditions required for this purpose are familiar to those skilled in the art and may be determined by routine experiments. Preferably, simultaneous desilylation and removal of the ester group —C(O)—R¹¹ can be achieved by treatment of a compound of formula (III) with the alkali metal base in a C₁-C₄-alkanol, such as methanol, at temperatures in the range of 20 to 50° C. The amount of alkali metal base, in particular alkali metal carbonate, is preferably 3 to 10 equivalents and especially 4 to 7 equivalents, based on the compound (III), i.e. in case of the alkali metal carbonate 1.5 to 5 mol, in particular 2 to 3.5 mol per mole of compound (III). The cleavage of the protective groups C(R²)₂ and OR³ can be achieved by analogy to the methods described under b.1).

The 2′-O-fucosyllactose obtained after removal of the protective groups can be purified by using conventional purification methods, such as chromatography or crystallization, optionally with the aid of additives, such as charcoal, silica or polyvinyl pyrrolidone. Typical conditions for the crystallization of 2′-O-fucosyllactose can be found in Chem. Ber. 1956, 11, 2513. Depending on the reaction conditions and the method of purification, the obtained 2′-O-fucosyllactose may contain lactose, e.g. in an amount of 1% to 20%, based on the weight of the product. Chemical purity of 2′-O-fucosyllactose, minus lactose, is usually at least 90%, in particular at least 95% or higher. However, lactose is not a problematic impurity, because the amount of lactose is not problematic for the use of 2′-O-fucosyllactose in food.

In particular, the method of the invention allows for producing 2′-O-fucosyllactose in a manner, such that, even before work-up, the amount of the undesirable β-isomer β-2′-O-fucosyllactose (=β-L-fucopyranosyl)-(1→2)-O-β-D-galactopyranosyl-(1→4)-D-glucopyranose) is so low that purification of the reaction product yields 2′-O-fucosyl-lactose which contains less than 1% by weight, in particular less than 0.5% by weight β-2′-O-fucosyllactose, based on the total amount of 2′-O-fucosyllactose. This was not possible so far. Contrary to the methods of prior art, the method of the invention does not require transition metal catalysts for hydrogenolytic cleavage of benzyl protective groups and, thus, the concentration of transition metals in the 2′-O-fucosyllactose obtainable by the method of the invention is frequently less than 1 ppm and in particular below the level of detection.

The compounds of the formula (I), wherein X is an S-bound radical, that are used in step a) of the method according to the invention may be prepared by the following sequence of reaction steps:
1) peracylation of L-fucopyranose;
2) introduction of an S-bound radical at the anomeric carbon atom;
3) removal of the acyl protecting groups; and
4) silylation of the hydroxyl groups in positions 2, 3 and 4 of the thiofucoside.

In step 1), L-fucopyranose is converted into the respective peracylated fucose in a manner known per se in the art, as described e.g. in P. G. M. Wuts et al., “Greene's Protecting Groups in Organic Synthesis”, 4th Edition, Wiley 2006, D. Lloyd et al., J. Org. Chem. 2014, 79, 9826-29, WO 2010/115934, WO 2010/115935 and the literature cited therein. The reaction is typically performed as depicted in Scheme 1.

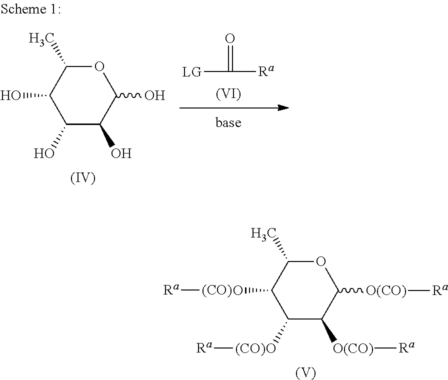

L-fucopyranose of formula (IV) is treated with the acylating reagent of the formula (VI), wherein LG is a suitable leaving group and the radical R^{a} is preferably a C₁-C₄-alkyl group or an optionally substituted phenyl group, in the presence of a base. The acylating reagent of the formula (VI) is usually a carboxylic acid or an activated derivative thereof, such as the corresponding anhydride or acyl chloride. Preferably, the acylating reagent (VI) is acetyl chloride, i.e. LG is Cl and R^{a} is methyl, acetyl anhydride, i.e. LG is CH₃C(O)O— and R^{a} is methyl, or benzoyl chloride, i.e. LG is Cl and R^{a} is phenyl, and in particular is acetyl anhydride. Accordingly, the compound of the formula (V) is preferably 1,2,3,4-O-tetra-acetyl-L-fucopyranose or 1,2,3,4-O-tetra-benzoyl-L-fucopyranose and in particular 1,2,3,4-O-tetra-acetyl-L-fucopyranose. The base used in the peracylation of step 1) is typically a tertiary amine, such as in particular pyridine. The reaction may be carried out in anhydrous inert solvents, such as chlorinated hydrocarbons, e.g. dichloromethane or dichloroethane, ethers, e.g. tetrahydrofuran or 1,4-dioxane. However, it is also possible to omit such a solvent, for example, if a suitable base, such as pyridine, is used in such an amount that it can also function as solvent. Step 1) is ordinarily carried out at temperatures in the range from −20° C. to 50° C. and preferably in the range from −10 to 30° C.

The reaction of step 2) is carried out in analogy to established procedures for preparing thioglycosides starting from the respective peracetylated saccharides, as described for example in the “Handbook of Chemical Glycosylation” edited by Alexei V. Demchenko, 2008, Wiley-VCH Verlag, Weinheim, Germany, D. Lloyd et al., J. Org. Chem. 2014, 79, 9826-29, WO 2010/115934, WO 2010/115935 and the literature cited therein. The reaction is typically conducted according to the route depicted in Scheme 2 by converting the peracylated fucose of formula (V) into the corresponding triacylated thiofucoside of the formula (VII).

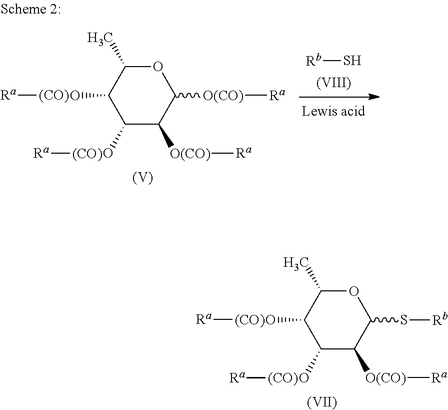

The peracylated fucose of formula (V) is reacted with the thiol of the formula (VIII), wherein the radical R^{b} is a radical R^{X1} or R^{X2} as defined herein above and is preferably methyl, ethyl or phenyl. The thiol (VIII) may be replaced by one of its suitable precursors, which are known from the art. Preferably, however, the thiol (VIII) is used in the reaction. The Lewis acid is generally selected from trimethylsilyl triflate, boron trifluoride diethyl etherate, tin(IV) chloride, titanium tetrachloride, iron(III) chloride, zirconium(IV) chloride, MoO₂Cl₂, and p-toluenesulfonic acid, with trimethylsilyl triflate and boron trifluoride diethyl etherate being preferred. The reaction typically takes place in an inert solvent, such as a chlorinated hydrocarbon, e.g. trichloromethane, dichloromethane and dichloroethane, or a ether, e.g. tetrahydrofuran or 1,4-dioxane. Step 2) is ordinarily carried out at temperatures in the range from −20° C. to 40° C. and preferably in the range from −10 to 25° C.

In step 3), the triacylated thiofucoside of the formula (VII) is deacylated via the reaction depicted in Scheme 3 to give the corresponding unprotected thiofucoside of the formula (IX). This conversion is performed in a manner known per se in the art, as described e.g. in P. G. M. Wuts et al., “Greene's Protecting Groups in Organic Synthesis”, 4th Edition, Wiley 2006, D. Lloyd et al., J. Org. Chem. 2014, 79, 9826-29, WO 2010/115934, WO 2010/115935 and the literature cited therein.

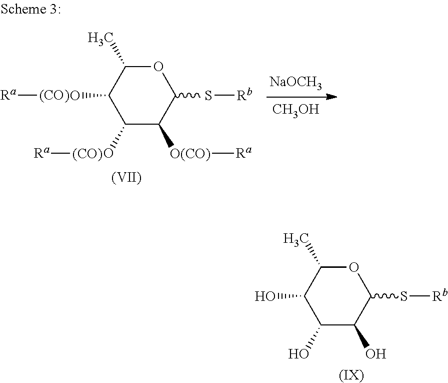

In order to remove the acyl protecting groups, the triacylated thiofucoside of the formula (VII) is generally treated with a base, in particular sodium methoxide in methanol, at temperatures in the range of 10 to 50° C.

In step 4), the thiofucoside of the formula (IX) is converted into the respective trisilylated thiofucoside of the formula (X) via the route depicted in Scheme 4.

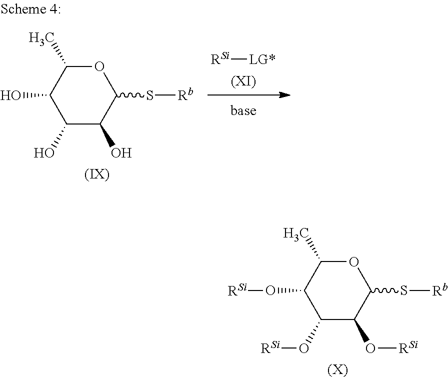

The thiofucoside of the formula (IX) is typically silylated by reacting it with a silylating reagent of the formula (XI), where the radical R^{Si} stands for a group —SiR¹²R¹³R¹⁴, as defined herein before, wherein R¹², R¹³ and R¹⁴ have the previously defined meanings and are especially methyl. The group LG* is a suitable leaving group, which in general is halogen, particularly chlorine. The reaction with the silylating reagent is preferably carried out in the presence of a base, such as in particular tertiary aliphatic amines, especially trimethylamine, or pyridine. The reaction temperature is usually in the range from −20 to 20° C., especially in the range from −5 to 5° C., e.g. at about 0° C. The conversion of step 4) ordinarily takes place in an aprotic solvent, particularly one having a low content of protic impurities, such as water, alcohols or acid. Preferred aprotic solvents are haloalkanes, such as dichloromethane, trichloromethane or dichloroethane, aromatic hydrocarbons, such as toluene and xylenes, dialkyl ethers, such as diethyl ether and diisopropyl ether, as well as cyclic ethers, such as tetrahydrofuran and dioxane.

Alternatively, the compounds of the formula (I), wherein X is an S-bound radical, may be prepared in analogy to the procedure disclosed in Y.-C. Ko et al., J. Am. Chem. Soc., 2014, 136 (41), 14425-31, by initially persilylating L-fucopyranose and subsequently introducing an S-bound radical at the anomeric carbon atom by the reaction with a suitable thiol derivative.

Compounds of the formula (II), where R¹ is a C(═O)—R¹¹ residue, are known, e.g. from the references cited at the outset, or from Tetrahedron Letters, 1981, 22 (50), 5007-5010, WO 2010/115934, WO 2010/115935 and Carbohydrate Research 88 (1981), 51-60, or may be prepared in analogy to the methods described therein.

Compounds of the formula (II), where R¹ is a SiR¹²R¹³R¹⁴ radical, may be prepared in a simple manner from the compounds of the formula (IIb) by selective silylation of the CH₂—OH group.

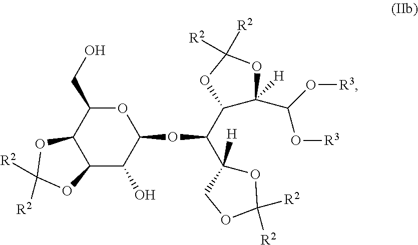

R² and R³ in formula (IIb) are as defined above, and particularly are defined as follows:

R² is in particular C₁-C₄-alkyl and especially methyl, or two R² residues attached to the same carbon atom are together 1,5-pentanediyland thus form a cyclohexane-1,1-diyl residue with the carbon atom to which they are attached. All R² residues are especially methyl.

R³ is particularly C₁-C₄-alkyl and especially methyl.

For the selective silylation, the compound of the formula (IIb) is typically reacted with a suitable silylating reagent, e.g. a compound of the formula SiXR¹²R¹³R¹⁴, where R¹², R¹³ and R¹⁴ are as defined previously and are especially methyl, and X is halogen, particularly chlorine. The reaction with the silylating reagent is preferably carried out in the presence of a base.

For the selective silylation, 0.9 to 2 mol, particularly 1 to 1.5 mol, especially about 1.1 mol of the silylating reagent, is typically used per mole of the compound of the formula (IIb).

In order for the reaction to proceed selectively, the reaction of (IIb) is preferably carried out in the temperature range from −40 to +40° C., particularly in the range from −20 to +20° C., especially preferably in the range from −5 to +5° C., e.g. at about 0° C.

Suitable bases are primarily amine bases, particularly secondary and tertiary amines, especially pyridine bases and tertiary aliphatic or cycloaliphatic amines. Suitable pyridine bases are, for example, pyridine, quinoline and C₁-C₆-alkyl-substituted pyridines, particularly mono—, di—and tri(C₁-C₆-alkyppyridines, such as 2,6-di(C₁-C₆-alkyppyridines and collidine. Suitable tertiary aliphatic or cycloaliphatic amines are tri(C₁-C₆-alkyl)amines, such as triethylamine, diisopropylmethylamine, tri-n-butylamine or isopropyldimethylamine, C₃-C₈-cycloalkyl-di(C₁-C₆-alkyl)amines, such as cyclohexyldimethylamine, N-(C₁-C₆-alkyl)piperidine, such as N-methylpiperidine and di(C₃-C₈-cycloalkyl)-C₁-C₆-alkylamines, such as biscyclohexylmethylamine.

The base is typically used in an amount of 0.9 to 2 mol, particularly in an amount of 1 to 1.5 mol per mole of the compound of the formula (IIb).

The compound of the formula (IIb) is reacted with the silylating reagent, generally in an inert organic solvent or diluent. Preference is given to aprotic solvents, particularly those having a low content of protic impurities, such as water, alcohols or acid. Preferred organic solvents are haloalkanes, such as dichloromethane, trichloromethane, dichloroethane, aromatic hydrocarbons, such as toluene and xylenes, dialkyl ethers, such as diethyl ether, diisopropyl ether, methyl tert-butyl ether, cyclic ethers, such as tetrahydrofuran or dioxane, dialkylamides of aliphatic carboxylic acids, such as dimethylformamide or dimethylacetamide and also alkyl nitriles, such as acetonitrile, and also mixtures of the abovementioned solvents. The solvent is preferably selected such that all constituents are present in dissolved form. The total concentration of the compound of the formulae (I) and (II) is preferably in the range of 5 to 50% by weight, particularly 10 to 40% by weight, based on the total weight of all reagents.

The compound of the formula (II), where R¹ is a SiR¹²R¹³R¹⁴ radical, can be worked-up by filtration, by extraction or in some cases by distillation.

The compounds of the formula (IIb) are known, e.g. from Carbohydrate Research, 212 (1991), pp. C₁-C₃; Tetrahedron Lett., 31 (1990) 4325; Carbohydrate Research, 75 (1979) C11; Carbohydrate Research, 88 (1981) 51; Chem. 5 (1999) 1512; WO 2010/070616, WO 2012/113404, WO 2010/115934 and WO 2010/115935 or may be prepared by the methods described therein.

As already mentioned, the 2′-O-fucosyllactose obtainable by the method according to the invention, in comparison to the known 2′-O-fucosyllactose, is characterized in that it does not comprise, or only comprises in much lower fractions, those impurities which cannot be removed. In particular, the 2′-O-fucosyllactose obtainable by the method according to the invention does not comprise significant amounts of impurities, particularly no impurities resulting from hydrogenation, which would be of concern for use in foodstuffs.

Accordingly, such a 2′-O-fucosyllactose is suitable itself as foodstuff and also as additive for foodstuff. Examples of foodstuff in which the 2′-O-fucosyllactose may be used are familiar to those skilled in the art, e.g. from the prior art cited at the outset. Here, this can take the form of compositions based on naturally occurring products, e.g. dairy products, and also artificially prepared formulations, for example, for dietary or medicinal nutrition. The latter can be ready-to-use formulations and can be used directly, or may take the form of concentrated formulations, e.g. liquid or semi-solid concentrates, or solid products, such as granules, flakes or powder which are converted into a ready-to-use formulation before use by addition of liquid, particularly water, or which are incorporated into a conventional foodstuff.

The concentrates and also the ready-to-use formulations can be solid, liquid or semi-solid formulations.

In particular, the foodstuffs, in which the 2′-O-fucosyllactose according to the invention is used, are foodstuff compositions for child nutrition, particularly in baby formula and especially infant formula.

In general, the foodstuffs, in which the 2′-O-fucosyllactose according to the invention is used, are solid, semi-solid or liquid foodstuff compositions, particularly semi-solid or especially liquid foodstuff compositions.

The foodstuff compositions, i.e. the ready-to-use foodstuff compositions and the concentrates, may be prepared in a manner known per se by incorporating the 2′-O-fucosyllactose obtainable according to the invention into a foodstuff formulation. This foodstuff formulation may comprise other nutrients, in addition to the 2′-O-fucosyl-lactose, and generally comprises at least one carrier suitable for foodstuff, wherein the latter may be solid, liquid or semi-solid. The carrier can be a foodstuff or a substance with nutritional value, or it may be a substance which itself has no nutritional value, e.g. dietary fiber or water.

The examples which follow serve to illustrate the invention.

The following abbreviations were used:

d: doublet

dd: doublet of doublets

ps-q: pseudo quartet

s: singlet

t: triplet

m: multiplet

mc: centered multiplet

CHCl₃: trichloromethane

CHE: cyclohexane

DCM: dichloromethane, preferably stabilized with amylene or without any stabilizer

DMF: dimethylformamide

of th: of theory

EE: ethyl acetate

BF₃*Et₂O: boron trifluoride diethyl etherate

MgSO₄: magnesium sulfate

MeOH: methanol

NaHCO₃: sodium hydrogencarbonate

RT: ambient temperature, about 22° C.

If not stated to the contrary, 2′-O-fucosyllactose refers to the alpha anomer.

HPLC analysis was performed using an Agilent Series 1200 and a Luna-NH2 column (3 μm; 250×4.6 mm, 100Å). The column was maintained at 35° C. and operated at 204 bar.

Acetonitrile/water 82.5/17.5 v/v was used as eluent; detection was with an RID detector. The flow rate was 1 mL/min, the run time 10 to 40 min. The sample volume was 5 μL.

For the sample preparation, 100 mg of sample were in each case dissolved in 10 mL of acetonitrile/water in a 50/50 ratio by volume.

The retention times of the individual compounds vary over time, the reasons for variation include column degradation and composition of the sample. Before measurement, reference samples of the starting materials in question, products in question and by-products in question were always measured to determine the actual retention time.

### EXAMPLES

### Preparation Example 1: Preparation of 1-thioethyl-2,3,4-tri-O-acetyl-L-fucopyranose

A solution of 47.1 g (0.14 mol) tetra-acetyl fucose in 200 mL of CHCl₃ were cooled to 0° C. and 13.3 g (0.21 mol) ethanethiol were then slowly added. Afterwards, 26.2 g (0.18 mol) of BF₃*Et₂O were added dropwise over a period of 15 min at 0° C. The reaction mixture was stirred for 24 h at RT and subsequently worked-up by diluting it with 100 mL of DCM. The thus obtained mixture was washed successively with 100 mL saturated solution of NaHCO₃ and 100 mL water. The organic phase was dried over MgSO₄, filtered and concentrated to dryness under reduced pressure. The residue was redissolved in EE/CHE 8:2 (v/v) and the solution filtrated through a pad of silica gel (250 mL). The fractions containing the product were collected and concentrated to dryness under reduced pressure. Yield: 32.6 g (69% of th.).

### Preparation Example 2: Preparation of 1-thioethyl-L-fucopyranose

32.6 g (0.097 mol) of 1-thioethyl-2,3,4-tri-O-acetyl-L-fucopyranose were dissolved in 350 ml of methanol at RT. After addition of 1.76 g of sodium methoxide, the resulting mixture was stirred for six days at RT. 2 g of an ion exchange resin (Amberlite IR 120, H⁺ form, strong acidic) were then added and the mixture stirred for 15 min at RT. Subsequently, the ion exchange resin was removed by filtration, and the filtrate was then concentrated to dryness under reduced pressure. The residue was taken up in 20 mL of n-heptane/EE 1:1 (v/v) and crystallized at 0° C. over a period of 2 h with stirring. Filtration and washing of the crystals with 10 mL of n-heptane afforded 16 g of the solid product.

### Example 1: Preparation of 1-thioethyl-2,3,4-tri-O-trimethylsilyl-L-fucopyranose

To a solution of 9.5 g (0.046 mol) 1-thioethyl-L-fucopyranose in 30 mL of DMF were added 15.23 g of trimethylamine and then add 16.7 g chlorotrimethylsilane dropwise at 0° C. After continued stirring for 4 h at 0° C., 75 mL of n-pentane were added, and then 50 mL of water were added dropwise. Subsequently, the organic phase was separated and washed with 3×25 mL of water, with 25 mL of a saturated NaCl solution and then concentrated to dryness under reduced pressure affording 17.3 g of the oily product.

¹H-NMR (CD₂Cl₂): δ 5.2 (d); 4.2 (d); 4.1 (ps-q); 3.9 (dd); 3.6-3.5 (m); 3.5 (ps-q); 3.3 (m); 2.6-2.3 (m); 1.1 (m); 1.0 (m); 0.5-0.0 (m)

### Preparation Example 3: Preparation of 1-thiophenyl-2,3,4-tri-O-acetyl-L-fucopyranose

To a solution of 24.1 g of tetra-acetyl fucose in 24 g toluene was added 10.2 g of thiophenol, and the solution was cooled to 0° C. 15.6 g of BF₃*Et₂O were then added dropwise, and the resulting mixture was stirred for 12 h. After extraction with 50 mL of a saturated NaHCO₃ solution, the organic layer was separated and washed with 2×50 mL water, dried over Na₂SO₄ and finally concentrated to dryness under reduced pressure affording 29 g of the crude title compound.

### Preparation Example 4: Preparation of 1-thiophenyl-L-fucopyranose

29 g of the crude 1-thiophenyl-2,3,4-tri-O-acetyl-L-fucopyranose were dissolved in 60 mL of methanol, and 1.6 g sodium methoxide were added. After stirring for 24 h at RT, 6 g of an ion exchange resin (Amberlite IRA 120, H⁺ form, strong acidic) were added and the mixture stirred for 15 min at RT. Subsequently, the ion exchange resin was removed by filtration, and the filtrate was then concentrated to dryness under reduced pressure to afford 17.5 g of the crude title compound.

### Example 2: Preparation of 1-thiophenyl-2,3,4-tri-O-trimethylsilyl-L-fucopyranose

To a suspension of 17.4 g of the crude 1-thiophenyl-L-fucopyranose in 50 mL of DMF were added 22.7 g of triethylamine (3.3 eq) and then slowly added 24.6 g (3.3 eq) of chlorotrimethylsilane at a temperature of −5 to 0° C. After continued stirring for 4 h at 0° C., 100 mL of n-pentane were added and then the reaction quenched by adding 70 mL of water. The aqueous layer was re-extracted with 50 mL of pentane, and the combined organic extracts were dried over Na₂SO₄. Concentration to dryness under reduced pressure afforded 10 g of the title compound, which was present in the form of a mixture of its α- and β-isomers, as revealed by ¹³C-NMR spectroscopy.

¹³C NMR (CD₂Cl₂, 126 MHz): δ (ppm) 136.04, 135.71, 132.12, 132.12, 130.33, 130.33, 129.47, 129.47, 129.07, 129.07, 127.02, 126.71, 90.25, 89.35, 77.42, 75.82, 75.67, 75.08, 72.70, 70.52, 69.42, 68.19, 17.32, 16.74, 0.78,0.78, 0.78, 0.69, 0.69, 0.69, 0.55, 0.55, 0.550.50, 0.50, 0.50, 0.28.0,28, 0.28,0.28, 0.28, 0.28.

### Preparation Example 5: Preparation of 4-O-(6-O-pivaloyl-3,4-isopropylidene-β-D-galactopyranosyl)-2,3; 5,6-bis-O-isopropylidene-D-glucose-dimethyl acetal

(Compound of formula (II-1): Compound of the formula (II), where R¹=C(═O)C(CH₃)₃, R²=CH₃, and R³=CH₃)

150 g (280 mmol) of 4-O-(3,4-isopropylidene-β-D-galactopyranosyl)-2,3;5,6-bis-O-isopropylidene-D-glucose-dimethyl acetal were charged in 190 mL of DCM. To this were added 57.19 g (565 mmol) of triethylamine. A solution of 49.56 g (411 mmol) of pivaloyl chloride in 35 mL of DCM was added over a period of 2 h, so that the temperature was kept below 27° C. Then, the reaction mixture was heated to reflux for 21 h (internal temperature: 49° C.). After cooling, the suspension was poured onto 410 mL of ice water, and the resulting mixture was stirred for 10 min. The organic phase was separated, and the aqueous phase was extracted once with 95 mL of DCM. The combined organic phases were successively washed with 95 mL of water, 95 mL of saturated NaCl solution, dried over 45 g of Na₂SO₄, and the solids were filtered off.

The filtrate was evaporated by means of rotary evaporation (40° C., 5 mbar) to afford 188.65 g of a product comprising 79.2% by weight of the title compound (89.2% of th.).

¹³C NMR (CD₂Cl₂, 500 MHz): δ (ppm) 178.24, 110.40, 110.30, 108.55, 105.65, 103.95, 79.36, 78.35, 78.12, 76.72, 75.62, 74.60, 73.60, 71.36, 64.99, 63.09, 56.51, 53.54, 38.98, 28.25, 27.35, 27.26, 27.26, 27.26, 26.68, 26.39, 25.82, 24.51.

### Example 3: Preparation of a Compound of the Formula (III), Wherein R¹=C(═O)C(CH₃)₃, R²=CH₃, R³=CH₃ and R^{Si}=trimethylsilyl

A solution of 0.3 mmol of the fucosyl acceptor of the formula (II-1) (see preparation example 3), 0.45 mmol of 1-thioethyl-2,3,4-tri-O-trimethylsilyl-L-fucopyranose and 0.45 mmol of N-iodosuccinimide in 25 mL in DCM were initially stirred with about 2 g of molecular sieve 4 Å for 1 h and then 0.05 mmol of trimethylsilyl triflate in 1 mL DCM were added at −25° C. Stirring was continued for 16 h at this temperature, and afterwards the reaction was terminated by quenching with 0.5 mL of trimethylamine. The mixture was diluted with 30 mL of DCM and the organic phase washed with 2×10 mL saturated sodium hydrogensulfite and 10 mL of water. After drying over 45 g of Na₂SO₄ and filtering off the solids, the filtrate was concentrated under reduced pressure. The raw product was redissolved in CHE/EE 5:1 (v/v), treated with 1% by volume of triethylamine and transferred onto the top of a silica gel column (dimension of the column: diameter d=9 cm, height h=37 cm, volume V˜2.3 L). The column was eluted under slight pressure. Product fractions were combined and concentrated at 45° C. and 5 mbar by means of rotary evaporation and then for 1 h by means of oil pump vacuo to afford the title compound.

¹³C NMR (CD₂Cl₂, 500 MHz): δ (ppm) 178.18, 110.26, 110.12, 108.96, 105.84, 101.80, 97.96, 80.26, 78.01, 77.56, 76.29, 75.94, 75.37, 75.23, 73.80, 71.23, 70.96, 69.25, 66.98, 65.98, 6278, 56.23, 5340, 39.01, 27.92, 27.41, 27.32, 27.27, 27.27, 27.27, 27.16, 26.35, 25.66, 17.08, 0.77, 0.77, 0.77, 0.62, 0.62, 0.62, 0.26, 0.26, 0.26.

### Example 4: Preparation of 2′-O-fucosyllactose

1 g of the obtained crude product obtained in Example 3 in 20 mL of methanol were treated with 0.2 g of K₂CO₃ and stirred for 16 h. Methanol was distilled off, then 20 mL of DCM were added, and the mixture was washed with 10 mL of water. The organic phase was dried over Na₂SO₄, concentrated to dryness and taken up in 25 mL of 0.5 N HCl, and the mixture was stirred for 8 h at RT. Subsequently, the mixture was neutralized by elution through a column charged with 5 mL of ion exchanger IMAC HP 661 followed by rewashing with 3×3 mL of water, and the combined aqueous phases were washed with 6 mL of DCM. After evaporation in vacuo, an amorphous product was obtained which was verified to be the title product by comparing its HPLC retention time with the one of an authentic sample of 2′—O-fucosyllactose (HPLC was carried out as described above).

1.-23. (canceled)

24. A method for preparing 2′-O-fucosyllactose comprising the steps of
a) reacting a fucose derivative of the general formula (I) 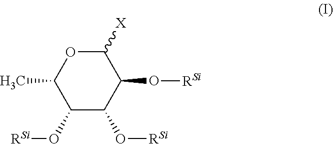
where
R^{Si} are the same or different radicals of the formula SiR^{a}R^{b}R^{c}, in which R^{a}, R^{b} and R^{c} are the same or different and are selected from C₁-C₈-alkyl, C₃-C₈-cycloalkyl, phenyl and C₃-C₈-cycloalkyl-C₁-C₄-alkyl;
X is selected from the group consisting of Br and S-bound radicals, with a compound of the general formula (II) 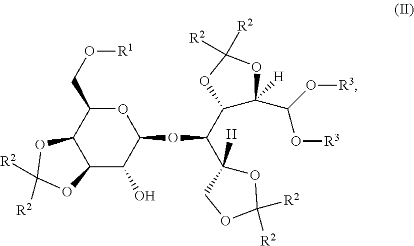
where
R¹ is a radical C(═O)—R¹¹ or a radical SiR¹²R¹³R¹⁴, in which R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from the group consisting of halogen, CN, NO₂, C₁-C₄-alkoxy, C₁-C₄-haloalkyl and C₁-C₄-haloalkoxy, and
R¹², R¹³ and R¹⁴ are the same or different and are selected from the group consisting of C₁-C₈-alkyl, C₃-C₈-cycloalkyl, phenyl and C₃-C₈-cycloalkyl-C₁-C₄-alkyl;
R² is the same or different and are C₁-C₈-alkyl, or two radicals R² attached to the same carbon atom together form a linear C₃-C₆-alkanediyl, which is unsubstituted or has 1 to 6 methyl groups as substituents;
R³ is the same or different and are C₁-C₈-alkyl or together form a linear C₁-C₄-alkanediyl, which is unsubstituted or has 1 to 6 methyl groups as substituents;
in the presence of an activating reagent; and
b) deprotecting the coupling product of the general formula (III) obtained in step a) 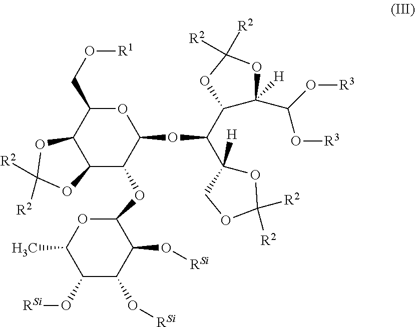
where R^{Si}, R¹, R² and R³ are as defined above;
to obtain 2′-O-fucosyllactose.

25. The method according to claim 24, wherein X in formula (I) is
—SCN, —S(O)ₙ—R^{X1} or —S—R^{X2}, where
n is 0, 1 or 2,
R_{X1} is aryl which is unsubstituted or optionally has 1 to 5 substituents selected from halogen, C₁-C₄-alkyl, C₁-C₄-alkoxy, C₁-C₄-haloalkyl and C₁-C₄-haloalkoxy, and
R^{X2} is selected from the group consisting of C₁-C₆-alkyl, C₁-C₆-haloalkyl, benzyl, wherein the phenyl moiety of benzyl is unsubstituted or optionally has 1 to 5 substituents selected from halogen, C₁-C₄-alkyl, C₁-C₄-alkoxy, C₁-C₄-haloalkyl and C₁-C₄-haloalkoxy, and 5- or 6-membered heterocyclyl, which bears a nitrogen atom in ortho position relative to the point of attachment and optionally a second heteroatom selected from O and S in the other ortho position, where heterocyclyl may optionally carry a fused benzyl moiety.

26. The method according to claim 24, wherein X in formula (I) is different from Br.

27. The method according to claim 25, wherein X in formula (I) is —S—R^{X1} or —S—R^{X2}, where
R^{X1} is phenyl, which is unsubstituted or optionally has 1, 2 or 3 substituents selected from halogen, C₁-C₄-alkyl and C₁-C₄-alkoxy, and
R^{X2} is selected from the group consisting of C₁-C₄-alkyl, 2-oxazolin-2-yl, 2-thiazolin-2-yl, benzoxazol-2-yl, benzothiazol-2-yl and pyridin-2-yl.

28. The method according to claim 24, wherein X in formula (I) is selected from the group consisting of methylthio, ethylthio and phenylthio.

29. The method according to claim 25, wherein the activating reagent is selected from the group consisting of the following reagents i) to xiii):
i) chloramine T,
ii) iodonium dicollidine perchlorate,
iii) dimethyl(methylthio)sulfonium triflate,
iv) N-bromosuccinimide,
v) N-iodosuccinimide,
vi) N-bromosuccinimide plus triflic acid,
vii) N-bromosuccinimide plus trimethylsilyl triflate,
viii) N-iodosuccinimide plus triflic acid,
ix) N-iodosuccinimide plus trimethylsilyl triflate,
x) bromine plus silver (I) triflate,
xi) diphenylsulfoxide plus triflic anhydride,
xii) iodine plus hexamethyldisilazane, and
xiii) copper (II) bromide plus tetra-(C₁-C₆-alkyl) ammonium bromide.

30. The method according to claim 29, wherein the activating reagent is selected from the group consisting of reagents vi), vii), viii) and ix).

31. The method according to claim 29, wherein the activating reagent is one of the reagents i) to v), that is employed in an amount of 0.05 to 2 molar equivalents per 1 mole of the compound of the formula (I).

32. The method according to claim 29, wherein the activating reagent is one of the reagents vi) to xiii), that is employed in such an amount, so that per 1 mole o f the compound of the formula (I) there are 1 to 2 molar equivalents, of the second mentioned component.

33. The method according to claim 24, where step a) comprises reacting the compound of formula (I), wherein the radical X is an S-bound radical different from Br, with bromine to obtain a compound of formula (I), wherein X is Br, followed by reacting the compound of formula (I), wherein X is Br, with the compound of formula (II) in the presence of the activating reagent.

34. The method according to claim 33, wherein the activating reagent is selected from the group consisting of alkali metal bromides, alkaline earth metal bromides and tetra-(C₁-C₆-alkyl) ammonium bromide.

35. The method according to claim 33, wherein the activating reagent is tetra-n-butyl ammonium bromide.

36. The method according to claim 35, wherein the activating reagent is used in an amount of 0.05 to 2 moles per mole of the compound of the formula (I).

37. The method according to claim 24, wherein the compound of the formula (I) and the compound of the formula (II) are reacted in a molar ratio (I):(II) in the range of 1:3 to 3:1.

38. The method according to claim 24, wherein the reaction of step a) is carried out in an aprotic solvent selected from the group consisting of dichloromethane, acetonitrile, DMF, toluene, THF, diethyl ether, dimethoxyethane, 1,4-dioxane and mixtures thereof.

39. The method according to claim 24, wherein the reaction of step a) is carried out at temperatures within the range of −40 to 60° C.

40. The method according to claim 24, wherein in step b)
b.1) the compound of the formula (III) is treated with water in the presence of an acid; or
b.2) the compound of the formula (III), in which R¹ is a radical SiR¹²R¹³R¹⁴, is firstly treated with a desilylating reagent, wherein a compound of the formula (IIIb) is obtained: 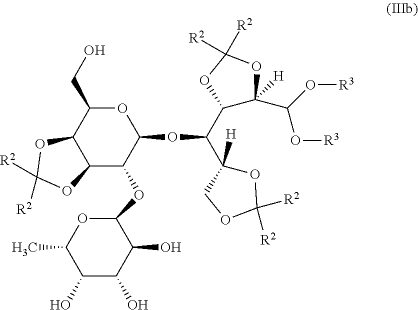
and subsequently the remaining protecting groups are removed by treating the compound of the formula (IIIb) with water in the presence of an acid; or
b.3) he compound of the formula (III), in which R¹ is a radical C(O)R¹¹, is firstly treated with a desilylating reagent, wherein a compound of the formula (IIIa) is obtained: 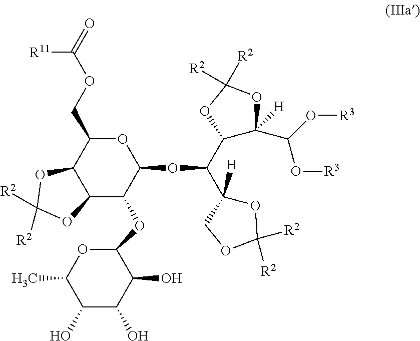
and subsequently the C(O)—R¹¹ group and the remaining protecting groups are successively removed; or
b.4) the protecting groups C(R²)₂ and OR³ are firstly removed from the compound of the formula (III), in which R¹ is a radical C(O)R¹¹, wherein a compound of the formula (IIIc) is obtained: 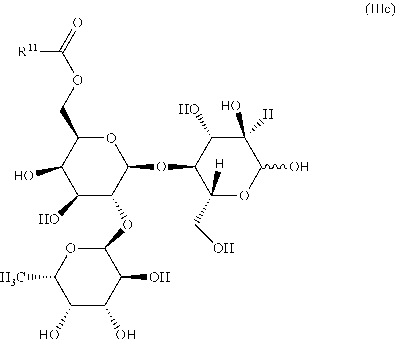
and the C(O)—R¹¹ group is subsequently removed, or
b.5) the compound of formula (III), wherein R¹ is a radical C(O)—R¹¹, is first treated with a C₁-C₄-alkanol and an alkalimetal base, whereby a compound of formula (IIIb) is obtained, and subsequently the remaining protecting groups are removed by treating the compound of the formula (IIIb) under acidic reaction conditions.

41. The method according to claim 24, wherein the radical R^{Si} in the formulae (I) and (III) is trimethylsilyl.

42. The method according to claim 24, wherein the radical R¹ in the formulae (II) and (III) is trimethylsilyl.

43. The method according to claim 24, wherein the radical R¹ in the formulae (II) and (III) is selected from the group consisting of acetyl, pivaloyl, benzoyl, 4-chlorobenzoyl and 4-methylbenzoyl.

44. The method according to claim 24, wherein the radical R² in the formulae (II) and (III) is methyl.

45. The method according to claim 24, wherein the radical R³ in the formulae (II) and (III) is methyl.

46. A fucose derivative of the general formula (I) 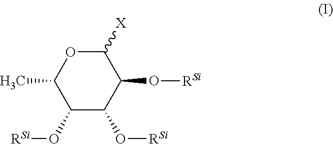
where
R^{Si} are the same or different radicals of the formula SiR^{a}R^{b}R^{c}, in which R^{a}, R^{b} and R^{c} are the same or different and are selected from C₁-C₈-alkyl, C₃-C₈-cycloalkyl, phenyl and C₃-C₈-cycloalkyl-C₁-C₄-alkyl;
X is selected from the group consisting of Br and S-bound radicals, namely —SCN, —S(O)ₙ—R^{X1} or —S—R^{X2}, where
n is 0, 1 or 2,
R^{X1} is aryl which is unsubstituted or optionally has 1 to 5 substituents selected from halogen, C₁-C₄-alkyl, C₁-C₄-alkoxy, C₁-C₄-haloalkyl and C₁-C₄-haloalkoxy, and
R^{X2} is selected from the group consisting of C₁-C₆-alkyl, C₁-C₆-haloalkyl, benzyl, wherein the phenyl moiety of benzyl is unsubstituted or optionally has 1 to 5 substituents selected from halogen, C₁-C₄-alkyl, C₁-C₄-alkoxy, C₁-C₄-haloalkyl and C₁-C₄-haloalkoxy, and 5- or 6-membered heterocyclyl, which bears a nitrogen atom in ortho position relative to the point of attachment and optionally a second heteroatom selected from O and S in the other ortho position, where heterocyclyl may optionally carry a fused benzyl moiety;
except for compounds of the formula (I), wherein
R^{Si} is trimethylsilyl, and X is —S-(4-methyl-phenyl),
R^{Si} is triethylsilyl, and X is —S-ethyl or —S(O)-phenyl, or
R^{Si} is tert-butyldimethylsilyl, and X is Br or —S-ethyl.

47. The fucose derivative according to claim 46, wherein R^{Si} in formula (I) is trimethylsilyl and X is selected from Br, methylthio, ethylthio and phenylthio.
